(19) United States
(12) Patent Application Publication
(10) Pub. No.: US 2026/0345226 A1
(43) Pub. Date: Nov. 5, 2026

(54) DIATHERMY SURGICAL TOOLS

(71) Applicant: Alcon Inc., Fribourg (CH)

(72) Inventors: Niels Abt, Winterthur (CH); Reto Grüebler, Greifensee (CH); Niccolo Maschio, Winterthur (CH); Christoph Siegenthaler, Eschenz (CH)

(21) Appl. No.: 19/637,237

(22) Filed: Apr. 2, 2026

Related U.S. Application Data
(60) Provisional application No. 63/798,079, filed on May 1, 2025.

(51) Int. Cl. A61B 18/14; A61B 18/00

(52) U.S. Cl. CPC A61B 18/1445; A61B 2018/00083; A61B 2018/00601; A61B 2018/00607; A61B 2018/0091; A61B 2018/1415; A61B 2218/007

(57) ABSTRACT

In certain embodiments, a surgical device comprises a diathermy electrical cable, an aspiration tube, at least one pneumatic tube, a handpiece, and a tip attached to the handpiece. The handpiece comprises a pneumatic actuator coupled to the pneumatic tube. The tip comprises an outer tube, a reciprocating inner tube, a conductor strip disposed on the outer tube, and an electrical insulating layer disposed between the outer tube and the conductor strip. The outer tube comprises a closed end and an outer port. The reciprocating inner tube comprises a cutting surface defining an open end. The reciprocating inner tube is disposed within the outer tube and is coupled to the pneumatic actuator and the aspiration tube. The outer tube and the conductor strip are coupled to the diathermy electrical cable. The outer tube is a first diathermy electrode, and the conductor strip is a second diathermy electrode.

### INTRODUCTION

The present disclosure relates to surgical systems. More particularly, the present disclosure relates to surgical systems and devices that combine diathermy and other surgical functions in a single surgical tool.

During an ophthalmic surgical procedure, the retina may begin bleeding into the vitreous humor of the eye. The blood may obstruct the view of the fundus (which includes the retina, macula, optic disc, fovea, and blood vessels), and cause the surgeon to pause the surgical procedure. Before continuing, the surgeon must remove the blood from the vitreous humor, determine the source of the bleeding, and then stop the bleeding from the retina. For example, the surgeon may insert an aspiration probe into the eye to aspirate the blood from the vitreous humor. After a clear view of the fundus has been restored, the surgeon may determine the source of the bleeding. The surgeon then removes the aspiration probe from the eye and inserts a diathermy probe to apply an electrical stimulus to the source of the bleeding. The diathermy probe stops the bleeding by coagulating the tissue at the source of the bleeding.

Similarly, the retina may begin bleeding into the vitreous humor of the eye due to the operation of a surgical tool, such as a vitrectomy probe, a forceps, a scissors, etc., and cause the surgeon to pause the surgical procedure. Before continuing, the surgeon must determine the source of the bleeding and then stop the bleeding from the retina. For example, the surgeon may remove the surgical tool from the eye and determine the source of the bleeding. The surgeon may then insert a diathermy probe to apply an electrical stimulus to the source of the bleeding to coagulate the tissue, remove the diathermy probe, and then insert the surgical tool to continue the procedure.

### SUMMARY

In certain embodiments, a surgical device comprises a diathermy electrical cable, a flexible aspiration tube, at least one flexible pneumatic tube, a handpiece, and a tip attached to the handpiece. The handpiece comprises a pneumatic actuator coupled to a distal portion of the flexible pneumatic tube and is configured to support a distal portion of the diathermy electrical cable, a distal portion of the flexible aspiration tube, and the distal portion of the flexible pneumatic tube. The tip comprises an outer tube, a reciprocating inner tube, a conductor strip disposed on the outer tube, and an electrical insulating layer disposed between the outer tube and the conductor strip. The outer tube comprises a passageway and a distal portion comprising a closed end and an outer port. The reciprocating inner tube comprises a passageway and a distal portion comprising a cutting surface defining an open end. The reciprocating inner tube is disposed within the passageway of the outer tube and is coupled to the pneumatic actuator and the distal portion of the flexible aspiration tube. The outer tube and the conductor strip are coupled to the distal portion of the diathermy electrical cable. The outer tube is a first diathermy electrode, and the conductor strip is a second diathermy electrode.

### BRIEF DESCRIPTION OF THE DRAWINGS

FIG. 1A illustrates an example surgical system, in accordance with certain embodiments of the present disclosure.

FIG. 1B illustrates an example surgical console, in accordance with certain embodiments of the present disclosure.

FIG. 1C illustrates a portion of the example surgical console, in accordance with certain embodiments of the present disclosure.

FIG. 2A depicts a first example aspirating diathermy probe, in accordance with certain embodiments of the present disclosure.

FIGS. 2B, 2C, 2D, and 2E depict views of the tip of the first example aspirating diathermy probe, in accordance with certain embodiments of the present disclosure.

FIG. 3A depicts a left side cross-sectional view of the distal end of a second example aspirating diathermy probe, in accordance with certain embodiments of the present disclosure.

FIG. 3B depicts a left side cross-sectional view of the distal end of a variation of the second example aspirating diathermy probe, in accordance with certain embodiments of the present disclosure.

FIG. 4A depicts a left side cross-sectional view of the distal end of a third example aspirating diathermy probe, in accordance with certain embodiments of the present disclosure.

FIG. 4B depicts a left side cross-sectional view of the distal end of a variation of the third example aspirating diathermy probe, in accordance with certain embodiments of the present disclosure.

FIGS. 5A and 5B depict a left side view and a front view (respectively) of the distal end of a fourth example aspirating diathermy probe, in accordance with certain embodiments of the present disclosure.

FIGS. 6A and 6B depict a left side view and a front view (respectively) of the distal end of a fifth example aspirating diathermy probe, in accordance with certain embodiments of the present disclosure.

FIGS. 7A, 7B, 7C, and 7D depict views of the tip of a sixth example aspirating diathermy probe, in accordance with certain embodiments of the present disclosure.

FIGS. 8A and 8B depict a left side view (partial cutaway) and a front view (respectively) of the tip of a seventh example aspirating diathermy probe, in accordance with certain embodiments of the present disclosure.

FIGS. 9A and 9B depict a left side view (partial cutaway) and a front view (respectively) of the tip of an eighth example aspirating diathermy probe, in accordance with certain embodiments of the present disclosure.

FIGS. 10A and 10B depict a left side view and a front view (respectively) of the tip of a ninth example aspirating diathermy probe, in accordance with certain embodiments of the present disclosure.

FIGS. 11A and 11B depict a left side view and a front view (respectively) of the tip of a tenth example aspirating diathermy probe, in accordance with certain embodiments of the present disclosure.

FIG. 12A depicts a first example diathermy vitrectomy probe, in accordance with certain embodiments of the present disclosure.

FIGS. 12B and 12C depict a left side view and a front view (respectively) of a first example diathermy vitrectomy probe tip, in accordance with certain embodiments of the present disclosure.

FIGS. 13A and 13B depict a left side view and a front view (respectively) of a second example diathermy vitrectomy probe tip, in accordance with certain embodiments of the present disclosure.

FIG. 14A depicts a first example diathermy forceps, in accordance with certain embodiments of the present disclosure.

FIG. 14B depicts a second example diathermy forceps, in accordance with certain embodiments of the present disclosure.

FIG. 14C depicts a left side view of a first example diathermy forceps tip, in accordance with certain embodiments of the present disclosure.

FIG. 15 depicts a left side view of a second example diathermy forceps tip, in accordance with certain embodiments of the present disclosure.

FIG. 16A depicts a first example diathermy scissors, in accordance with certain embodiments of the present disclosure.

FIG. 16B depicts a second example diathermy scissors, in accordance with certain embodiments of the present disclosure.

FIG. 16C depicts a left side view of a first example diathermy scissors tip, in accordance with certain embodiments of the present disclosure.

FIG. 17 depicts a left side view of a second example diathermy scissors tip, in accordance with certain embodiments of the present disclosure.

### DETAILED DESCRIPTION

Embodiments of the present disclosure will now be described with reference to the figures, in which like reference numerals refer to like parts throughout.

During an ophthalmic surgical procedure, the retina may begin bleeding into the vitreous humor of the eye, as described above. In order to stop the bleeding, the surgeon may aspirate the blood from the vitreous humor using an aspiration probe (or handpiece), determine the source of the bleeding, exchange the aspiration probe for a diathermy probe, and then coagulate the source of the bleeding using the diathermy probe. The surgeon may also use a combined irrigation/aspiration (I/A) probe in order to maintain the volume of the anterior chamber and a stable intraocular pressure (IOP) using an irrigation fluid.

The surgeon may need to exchange the aspiration probe and the diathermy probe several times until the bleeding is under control. For example, the bleeding may not stop completely after the initial coagulation, and another sequence of aspiration and coagulation may be needed. In another example, the bleeding may stop after the initial coagulation and then begin again at the same location on the retina after the surgeon resumes the surgical procedure. In a further example, the bleeding may stop after the initial coagulation and then begin at a different location on the retina after the surgeon resumes the surgical procedure.

The sequence of aspiration, determination, and coagulation includes inserting the aspiration probe into the eye, aspirating the blood, removing the aspiration probe from the eye, determining the source of the bleeding, inserting the diathermy probe into the eye, coagulating the source of the bleeding, and removing the diathermy probe from the eye.

Embodiments of the present disclosure combine the functions of aspiration and coagulation into a single aspirating diathermy device (also known as a probe, a handpiece, etc.) which advantageously eliminates the need for the surgeon to change back and forth between separate aspiration and diathermy probes. Importantly, the bleeding may be stopped by coagulation even before the view of the fundus is fully restored by aspiration. In other words, the aspirating diathermy probe advantageously provides simultaneous or parallel aspiration and coagulation.

Embodiments of the present disclosure also combine diathermy with other surgical tools, such as vitrectomy probes, forceps, scissors, etc.

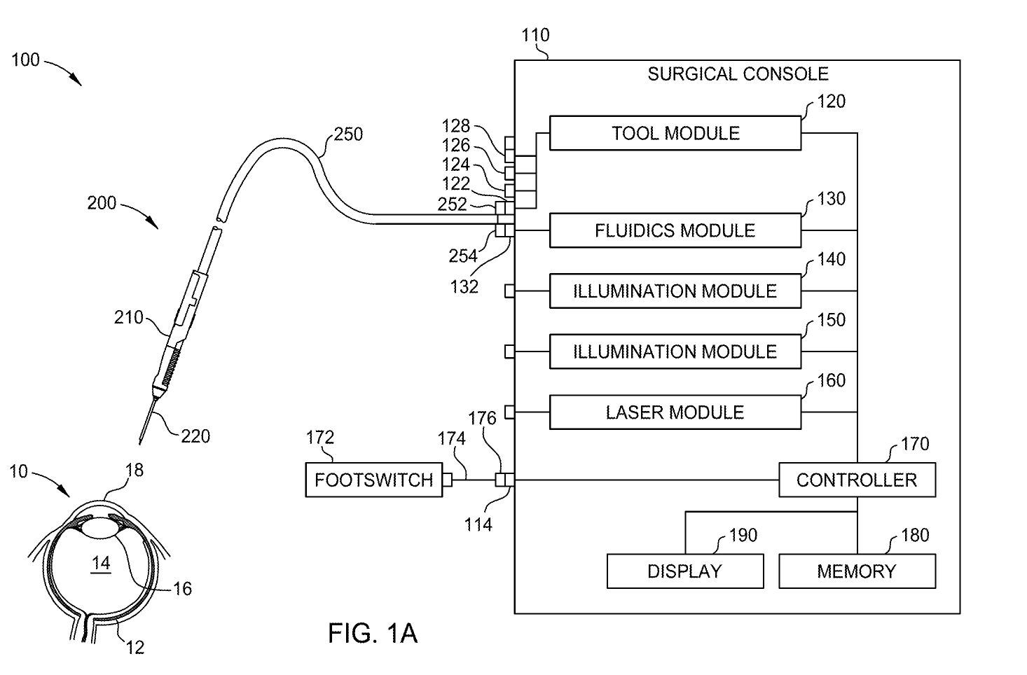

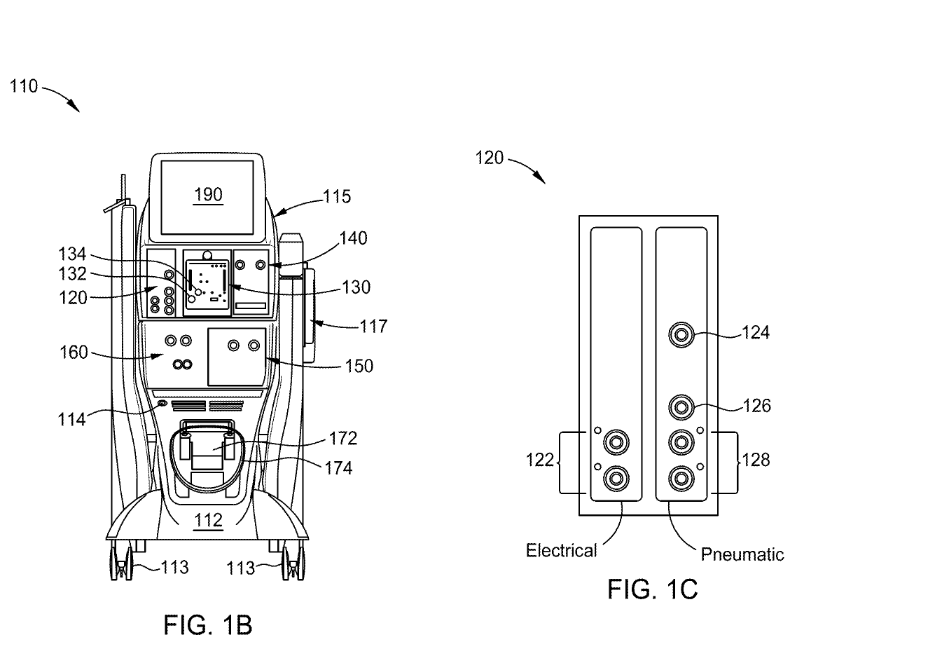

FIG. 1A illustrates an example surgical system 100 for performing an ophthalmic surgical procedure on a patient’s eye 10, in accordance with certain embodiments of the present disclosure. FIG. 1B illustrates an example surgical console 110, while FIG. 1C illustrates a portion of the example surgical console 110, in accordance with certain embodiments of the present disclosure.

Generally, the surgical system 100 may be used in a variety of ophthalmic surgical procedures that are performed on the patient’s eye 10, such as glaucoma surgery, vitreoretinal surgery, cataract surgery, etc. Additionally, the surgical system 100 may be configured to support a particular ophthalmic surgical procedure on the retina 12, the vitreous humor 14, the lens 16, the cornea 18, etc.

In certain embodiments, the surgical system 100 includes, among other things, a surgical console 110, a footswitch 172, and a surgical tool, such as an aspirating diathermy probe 200.

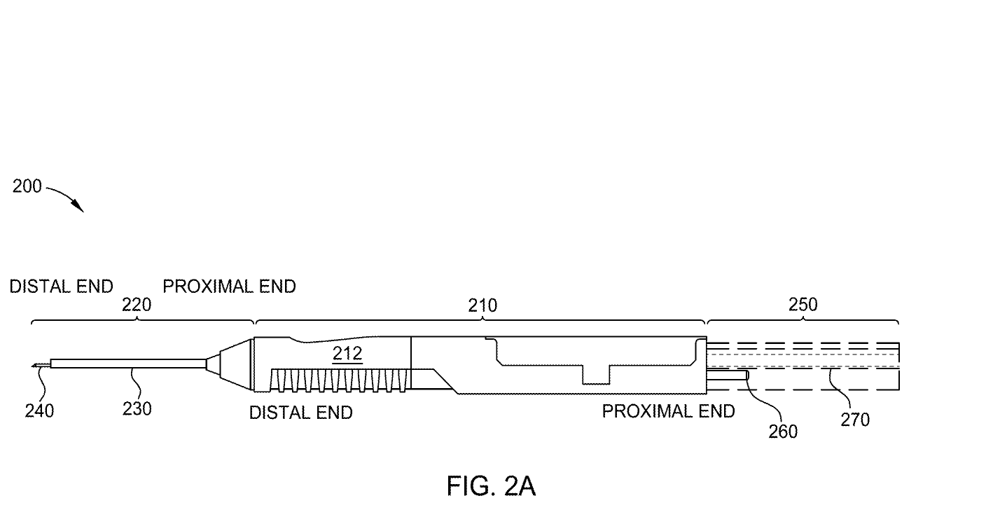

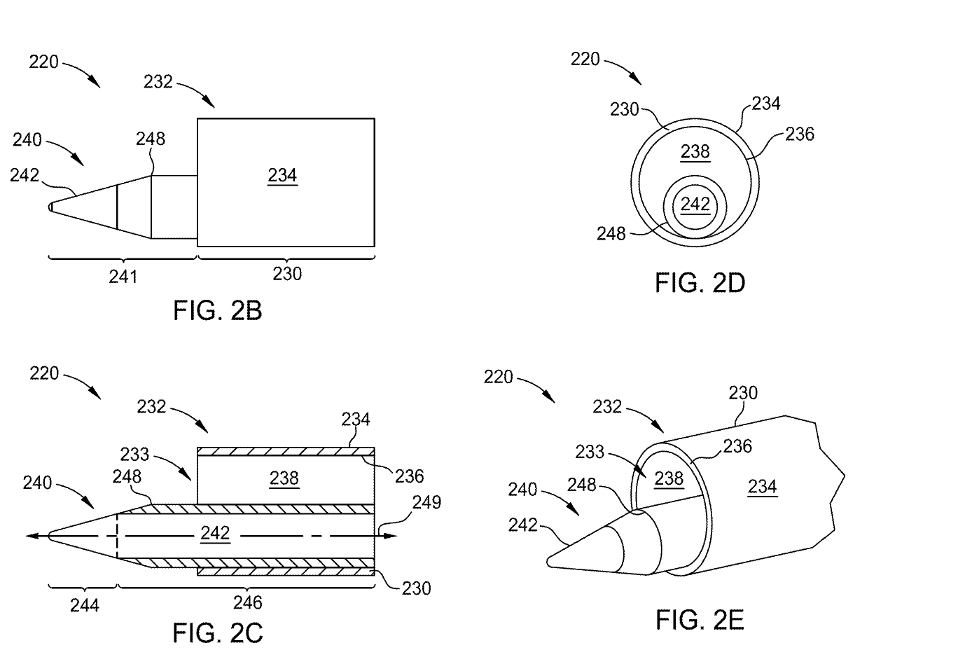

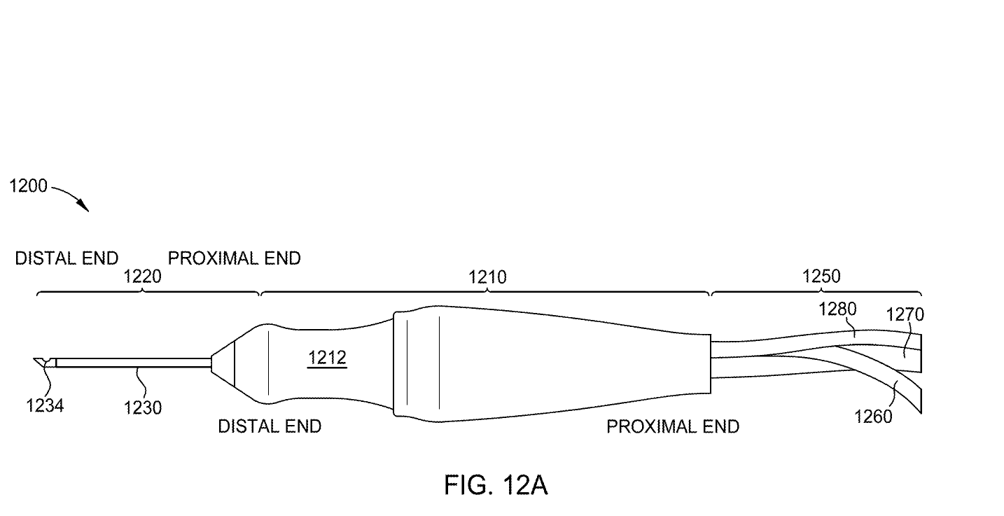

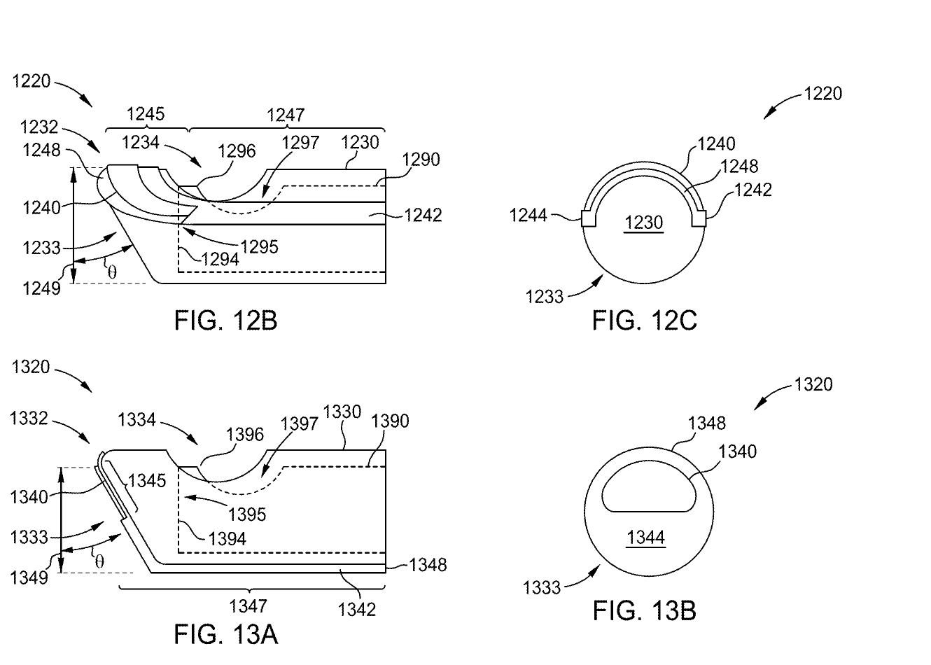

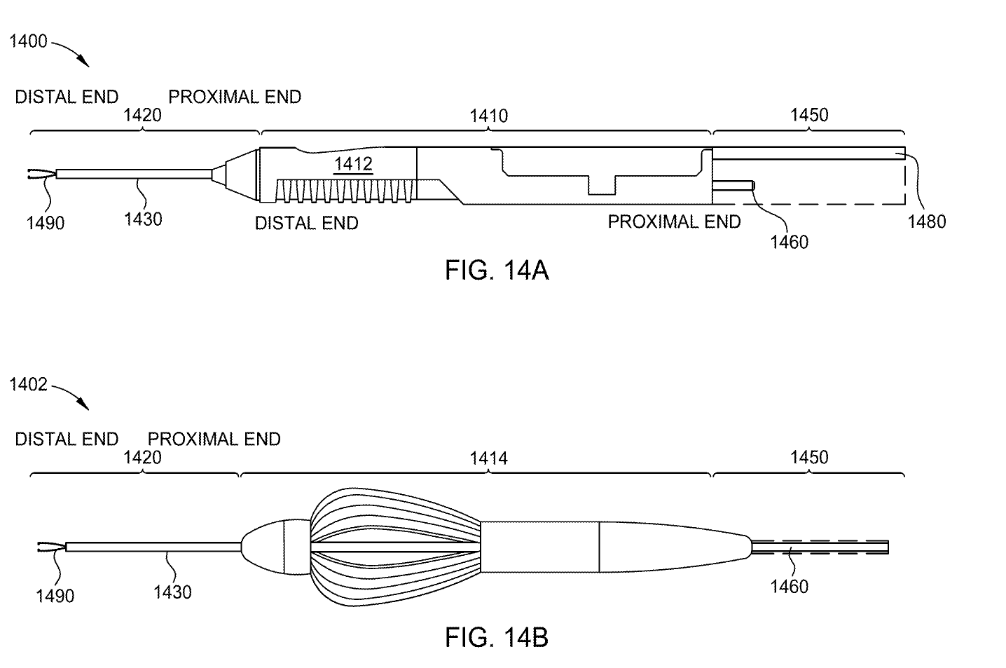

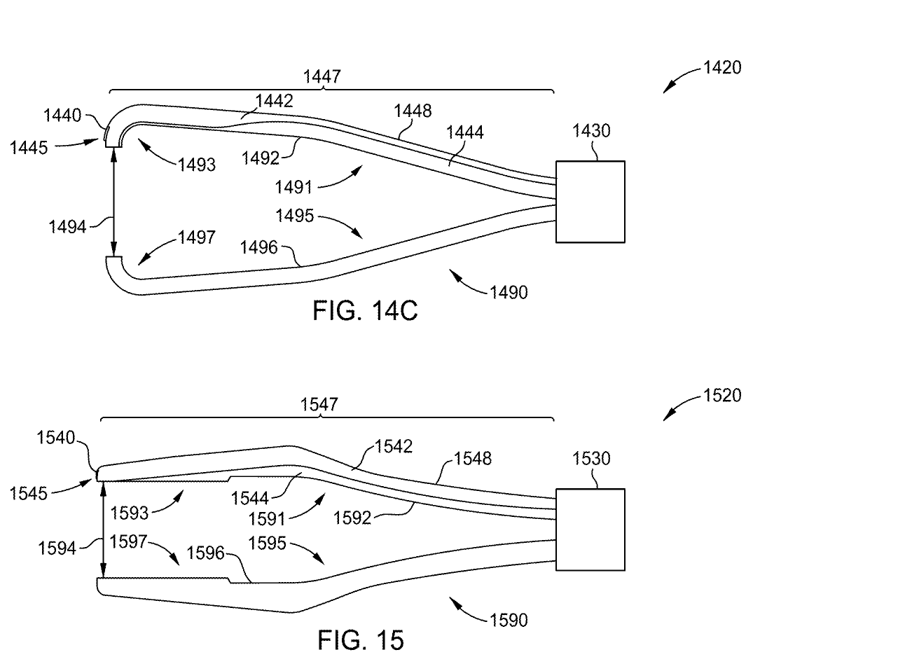

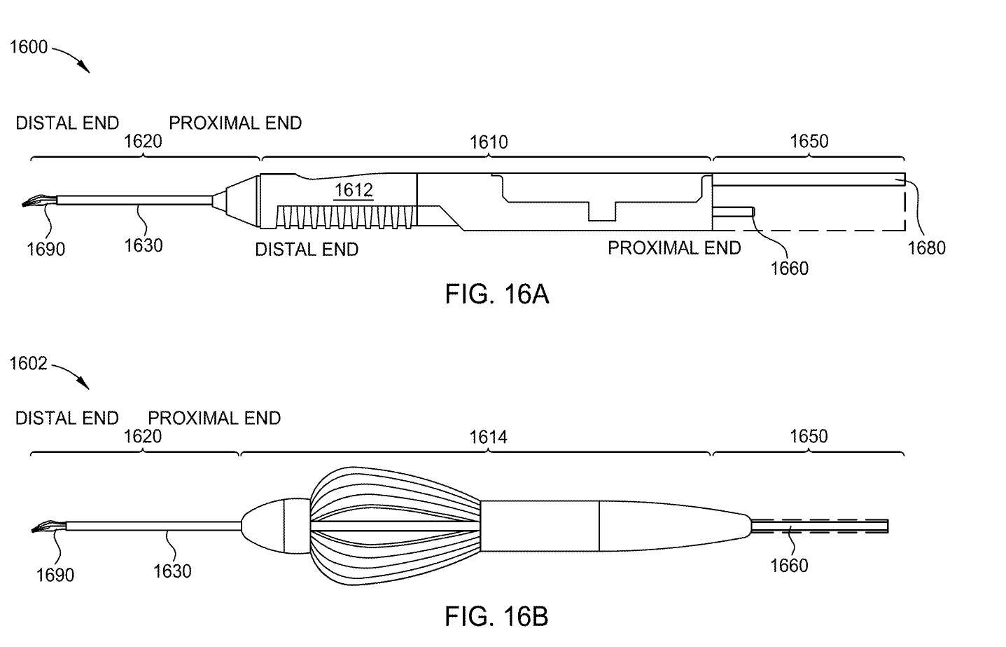

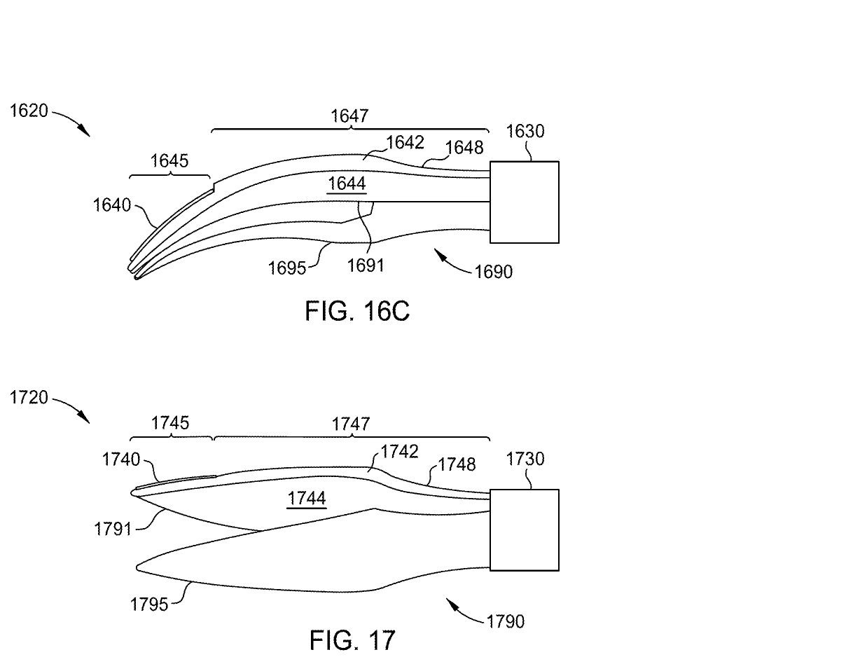

The aspirating diathermy probe 200 includes a handpiece 210, a tip 220, and a cable 250 with an electrical connector 252 and a fluidic connector 254. Embodiments of the aspirating diathermy probe 200 are discussed below with respect to FIGS. 2A - 11B. The surgical system 100 may also include other surgical tools, such as a diathermy vitrectomy probe 1200 (discussed below with respect to FIGS. 12A, 12B, 12C, 13A, and 13B), a diathermy forceps 1400, 1402 (discussed below with respect to FIGS. 14A, 14B, 14C, and 15), a diathermy scissors 1600, 1602 (discussed below with respect to FIGS. 16A, 16B, 16C, and 17), etc.

The surgical console 110 may include, among other things, a tool module 120, a fluidics module 130, an illumination module 140, another illumination module 150, a laser module 160, a controller 170, a memory 180, and a touchscreen display 190. An electrical cable 174 is coupled to the footswitch 172 and includes an electrical connector 176 that is configured to connect to the footswitch electrical connector 114. As depicted in FIG. 1B, the surgical console 110 may also include a base 112 with wheels 113, a housing 115, and an articulating instrument tray 117. The surgical console 110 may also include additional tools and subsystems to facilitate the performance of the ophthalmic surgical procedure. One example of a surgical console configured for performing anterior and posterior segment ophthalmic surgery is the Constellation® Vision System available from Alcon Laboratories, Inc., Fort Worth, Texas.

The tool module 120 includes the electrical and pneumatic control systems and power sources for the surgical tools (e.g., probes, handpieces, etc.) that may be used by the surgeon during the ophthalmic surgical procedure. The front panel of the tool module 120 includes electrical and pneumatic connectors that couple the electrical cables and pneumatic tubes of the surgical tools to the respective electrical and pneumatic control systems and power sources. In certain embodiments, the tool module 120 supports aspirating diathermy probes, pneumatic forceps, pneumatic scissors, pneumatic vitrectomy probes, etc. The tool module 120 may include diathermy electrical connectors 122, a forceps pneumatic connector 124, a scissors pneumatic connector 126, and vitrectomy probe pneumatic connectors 128.

The tool module 120 includes a diathermy electrical power supply that provides a variable output voltage that is selectable by the surgeon. In certain embodiments, the surgeon may select an output power control setting between 0 and 100 (%), which may provide, for example, between 0 W (Watts) and about 10 W of electrical power into a, for example, 75 Ohm load. (Other power and other resistances are also contemplated). In the example provided above, the relationship between the output power control setting and the output power may be generally linear, so an output power control setting of 50 % provides about 5 W of output power. Diathermy electrical connectors 122 are coupled to the diathermy electrical power supply. The diathermy electrical connectors 122 include two electrical terminals that support bipolar (i.e., 2 electrode) diathermy probes (such as aspirating diathermy probe 200), and each electrode is coupled to an electrical terminal when the diathermy probe is connected to the diathermy electrical connectors 122. The controller 170 controls the output voltage based on the operation of the footswitch 172 by the surgeon.

The diathermy electrical connectors 122 are coupled to the electrical connector 252 when the aspirating diathermy probe 200 is connected to the surgical console 110.

The tool module 120 also includes one or more pneumatic power sources, such as a constant air pressure source, a pulsed air pressure source, etc., that provide air pressure to power one or more pneumatic tools, such as a pneumatic forceps, a pneumatic scissors, a vitrectomy probe, etc. The pneumatic connectors 124, 126, 128 are coupled to the pneumatic power source(s), and each pneumatic connector provides an air supply to a different pneumatic tool. In some embodiments, an external air pressure source may be coupled to the surgical console 110 to provide compressed air to the tool module 120.

The fluidics module 130 includes the fluidic control systems and sources (such as irrigating fluid sources, vacuum sources such as aspiration pumps, etc.) for the surgical tools that may be used by the surgeon during the ophthalmic surgical procedure. The front panel of the fluidics module 130 includes fluidic connectors that couple the fluidic tubes of the surgical tools to the respective fluidic control systems and sources. In certain embodiments, the fluidics module 130 supports aspirating diathermy probes 200, aspiration probes, irrigation probes, I/A probes, etc. As depicted in FIG. 1B, the fluidics module 130 includes at least one aspiration port 132 and an irrigation port 134.

The aspiration port 132 is coupled to the fluidic connector 254 when the aspirating diathermy probe 200 is connected to the surgical console 110.

In some embodiments, the fluidics module 130 may be configured to receive a consumable cassette that is configured to regulate the irrigating fluid, aspirate debris, monitor irrigation and aspiration pressure, and deposit the debris in a sealed drainage bag for disposal. The consumable cassette includes the fluidic connectors, such as the aspiration port 132, the irrigation port 134, etc.

The illumination module 140 is configured to generate light that is formed into one or more illumination light beams. In certain embodiments, the illumination module 140 includes a xenon arc lamp that is coupled to two independently controlled optical ports. An illumination probe may be coupled to each optical port using a fiber optical cable. The controller 170 may independently control the light emitted by each optical port.

In some embodiments, the illumination module 140 includes three continuous wave (CW) lasers that generate laser light beams in the red, green, and blue spectrums in response to control signals provided by the controller 170. The red, green, and blue laser light beams are optically combined to form a white (or pseudo-white) illumination light beam (or some other user- or system- selected color) in the visible spectrum (e.g., about 380 nm (nanometers) to 750 nm). Other visible spectrum (color) combinations may also be used. In some other embodiments, the illumination module 140 includes three light emitting diodes (LEDs) that generate light beams in the red, green, and blue spectrums in response to control signals provided by the controller 170. The red, green, and blue light beams are optically combined to form a white (or pseudo-white) illumination light beam (or some other user- or system- selected color) in the visible spectrum. In some embodiments, the illumination module 140 includes a single CW laser or LED that generates a monochromatic light beam, such as a light beam in the red spectrum, the green spectrum, the blue spectrum, etc.

The illumination module 150 is similar to the illumination module 140 and is configured to generate light that is formed into one or more illumination light beams. In certain embodiments, the illumination module 140 includes a xenon arc lamp that is coupled to two independently controlled optical ports. An illumination probe may be coupled to each optical port using a fiber optical cable. In some embodiments, the illumination module 150 may include lasers, LEDs, etc., as described above with respect to the illumination module 140.

The laser module 160 may include a diode laser that is configured to generate a laser treatment beam for various ophthalmic surgical procedures, such as retinal and macular photocoagulation, iridotomy and anterior segment procedures, trans-scleral cyclophotocoagulation, laser trabeculoplasty, trans-scleral retinal photocoagulation, and other laser treatments.

The controller 170 is coupled to the footswitch electrical connector 114, the memory 180, and the touchscreen display 190. The controller 170 may be a microcontroller, a microprocessor, a programmable logic controller (PLC), a field programmable gate array (FPGA), an application specific integrated circuit (ASIC), etc. Generally, the controller 170 is configured to control the configuration and operation of the surgical console 110 through user inputs received from the footswitch 172 and the touchscreen display 190.

The footswitch 172 may be used to control both the electrical (diathermy) and fluidic (aspiration) functions of the aspirating diathermy probe 200. The footswitch 172 includes a number of switches that are configured by the controller 170 to perform various functions during the surgical procedure, such as a foot pedal (or treadle), a left horizontal/vertical switch, a right horizontal/vertical switch, a left heel switch, and a right heel switch. Generally, the foot pedal may be used to provide control of a parameter (such as aspiration vacuum) with the foot pedal’s angle of depression proportional to the parameter output. In some modes of operation of the surgical console 110, the foot pedal may be used to provide on/off control of a function (such as diathermy), with any depression resulting in a fixed output of its parameter.

The display 190 may include a touchscreen that presents different user interface screens to the surgeon. The controller 170 generates each user interface screen, which may include multiple windows, panels, tabs, menus, etc., each of which may include buttons, icons, diagrams, schematics, etc. The touchscreen display 190 accepts user input, which is provided to the controller 170 to configure and control the various functions of the surgical console 110.

FIG. 2A depicts the aspirating diathermy probe 200, in accordance with certain embodiments of the present disclosure.

In certain embodiments, the aspirating diathermy probe 200 includes, among other things, a handpiece 210, a tip 220, and a cable 250 that includes a diathermy electrical cable 260 and a flexible aspiration tube 270.

The handpiece 210 has an outer gripping surface 212 for the surgeon and includes a passageway (not visible) configured to support the distal portions of the diathermy electrical cable 260 and the flexible aspiration tube 270. The passageway extends from the proximal end of the handpiece 210 to the tip 220 attached to the distal end of the handpiece 210.

The tip 220 includes an aspiration tube 230 and an inner needle 240 disposed within the aspiration tube 230.

The aspiration tube 230 is made from electrically conductive material. The aspiration tube 230 is fluidically coupled to the flexible aspiration tube 270 and electrically coupled to one of the insulated conductors of the diathermy electrical cable 260. The aspiration tube 230 includes a passageway through which aspirated material may be removed, and functions as one of the electrodes for bipolar diathermy.

The inner needle 240 is electrically coupled to the other insulated electrical conductor of the diathermy electrical cable 260. The inner needle 240 has an insulated electrode that is made from an electrically conductive material, such as tungsten, copper, silver, stainless steel, etc., and functions as the other electrode for bipolar diathermy.

The cable 250 includes a diathermy electrical cable 260 connected to the electrical connector 252 (FIG. 1A), and a flexible aspiration tube 270 connected to the fluidic connector 254 (FIG. 1A). The diathermy electrical cable 260 includes two insulated electrical conductors, such as insulated copper wires, insulated tungsten wires, etc. Each insulated electrical conductor is coupled to an electrical terminal in the electrical connector 252.

In certain embodiments, the tip 220 is removable and includes a fluidic connector to couple the aspiration tube 230 to the flexible aspiration tube 270, and electrical connectors to couple the aspiration tube 230 and the inner needle 240 to the insulated electrical conductors of the diathermy electrical cable 260.

In some embodiments, the cable 250 includes a flexible irrigating fluid tube, and the tip 220 includes an irrigating fluid tube to deliver irrigating fluid to the eye 10. The flexible irrigating fluid tube is coupled to the irrigating fluid tube, and to a fluidic connector at the proximal end of the cable 250. The fluidic connector may be coupled to the irrigation port 134. Alternatively, the aspiration tube 230 may be coupled to a valve that selects between the flexible irrigating fluid tube and the flexible aspiration tube 270. The footswitch 172 may also be used to control the irrigation function of the aspirating diathermy probe 200.

FIGS. 2B, 2C, 2D, and 2E depict views of the tip 220 of the aspirating diathermy probe 200, in accordance with certain embodiments of the present disclosure.

More particularly, FIG. 2B depicts a left side view of the distal end of the tip 220, FIG. 2C depicts a left side cross-sectional view of the distal end of the tip 220, FIG. 2D depicts a front view of the distal end of the tip 220, and FIG. 2E depicts a perspective view of the distal end of the tip 220.

The aspiration tube 230 has a distal end 232 that defines an opening 233 through which the aspirated material is received, an outer surface 234, and an inner surface 236 that defines a passageway 238 through which the aspirated material is removed. The proximal end of the aspiration tube 230 also defines an opening that is fluidically coupled to the flexible aspiration tube 270. The proximal end of the aspiration tube 230 is also electrically coupled to one of the insulated electrical conductors of the diathermy electrical cable 260. Generally, a distal portion of the outer surface 234, the edge of the aspiration tube 230, and a distal portion of the inner surface 236 provide one electrode surface for bipolar diathermy.

The inner needle 240 includes a conductor 242 and an insulating layer 248. The conductor 242 is made from an electrically conductive material, such as tungsten, copper, silver, stainless steel, etc., and the insulating layer 248 is made from an electrically insulating material, such as Polyvinyl Chloride (PVC), nylon, etc. The proximal end of the conductor 242 is electrically coupled to the other insulated electrical conductor of the diathermy electrical cable 260.

In certain embodiments, the conductor 242 has a conical tip 244 at the distal end, and a central core 246 that extends from the conical tip 244 to the proximal end of the conductor 242. The insulating layer 248 covers the central core 246, and the conical tip 244 provides the other electrode surface for bipolar diathermy.

The inner needle 240 is generally disposed within the passageway 238, with the distal end 241 of the inner needle 240 extending out of the passageway 238 past the distal end 232 of the aspiration tube 230. In certain embodiments, the insulating layer 248 may abut a portion of the inner surface 236 in a lower portion of the passageway 238 (as depicted). In other embodiments, the insulating layer 248 may abut a portion of the inner surface 236 in a left portion, a right portion, or an upper portion of the passageway 238. In some embodiments, the inner needle 240 may be centrally disposed within the passageway 238 such that the insulating layer 248 does not touch the inner surface 236.

In some embodiments, the inner needle 240 may translate along a longitudinal axis 249 from an extended position (as depicted) to a retracted position in which the conical tip 244 is disposed within the passageway 238. The retracted position prevents the inner needle 240 from contacting the tissue within the eye 10 and advantageously allows the opening 233 of the aspiration tube 230 to be positioned more closely to any particular location in the eye 10 by the surgeon. The inner needle 240 may be attached to an actuator (such as a slide, a toggle, etc.) on the handpiece to allow the surgeon to extend and retract the inner needle 240.

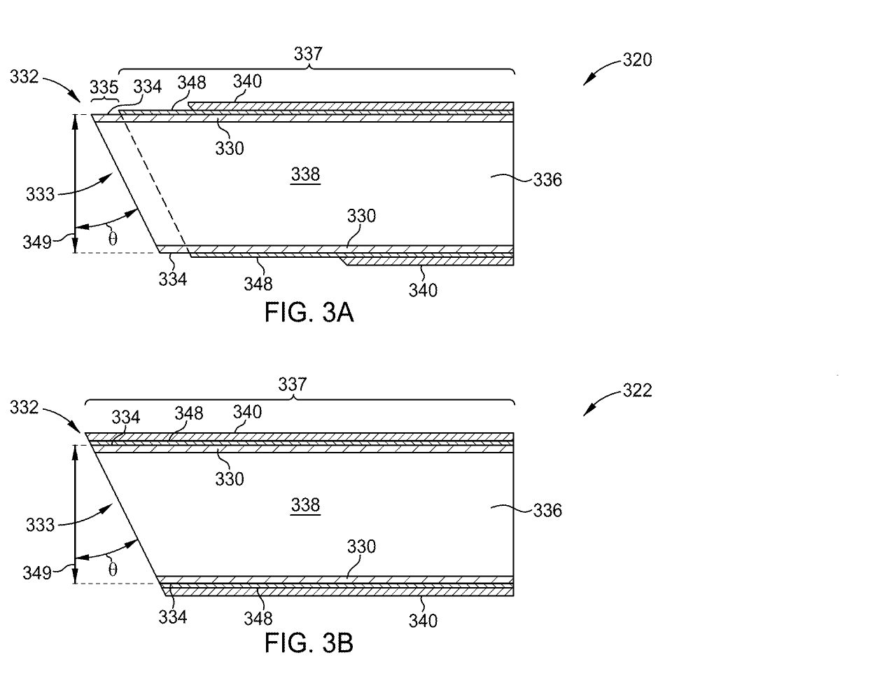

FIG. 3A depicts a left side cross-sectional view of the distal end of a tip 320 of a second example aspirating diathermy probe, in accordance with certain embodiments of the present disclosure. Generally, the second example aspirating diathermy probe includes the same components as the aspirating diathermy probe 200, with the tip 220 replaced by the tip 320.

In certain embodiments, the tip 320 includes, among other things, an aspiration tube 330, an outer tube 340, and an electrical insulating layer 348 disposed between the aspiration tube 330 and the outer tube 340. The distal end 332 of the aspiration tube 330 forms an angle θ with respect to the elevation axis 349 and defines an opening 333.

The aspiration tube 330 is made from an electrically conductive material. The aspiration tube 330 has an outer surface 334, and an inner surface 336 that defines an inner passageway 338 through which the aspirated material is removed. The proximal end of the aspiration tube 330 also defines an opening that is fluidically coupled to the flexible aspiration tube 270. The proximal end of the aspiration tube 330 is also electrically coupled to one of the insulated electrical conductors of the diathermy electrical cable 260. The aspiration tube bfunctions as one of the electrodes for bipolar diathermy.

The electrical insulating layer 348 covers most of the outer surface 334 (such as the insulated section 337) except for an exposed section 335 and the edge of the aspiration tube 330. The electrical insulating layer 348 is made from an electrically-insulating material, such as Polyvinyl Chloride (PVC), nylon, etc. Generally, the exposed section 335 of the outer surface 334, the edge of the aspiration tube 330, and a distal portion of the inner surface 336 provide one electrode surface for bipolar diathermy. The electrical insulating layer 348 may be applied to the aspiration tube 330 using various methods, such as by jet printing, etc. Generally, the thickness of the electrical insulating layer 348 may be about 4 to 6 microns, such as 5 microns. Other thicknesses may also be used, such as 6 to 10 microns, etc.

The outer tube 340 is electrically coupled to the other insulated electrical conductor of the diathermy electrical cable 260. The outer tube 340 is made from an electrically conductive material, and functions as the other electrode for bipolar diathermy. The outer tube 340 does not extend to the end of the aspiration tube 330.

FIG. 3B depicts a left side cross-sectional view of the distal end of a tip 322 of a variation of the second example aspirating diathermy probe, in accordance with certain embodiments of the present disclosure.

Generally, the tip 322 includes the same components as the tip 320. However, the insulating layer 348 covers all of the outer surface 334, which extends the insulated section 337 to the end of the aspirating tube 330 and removes the exposed section 335. The edge of the aspiration tube 330 and a distal portion of the inner surface 336 provide one electrode surface for bipolar diathermy. The outer tube balso extends to the end of the aspiration tube 330.

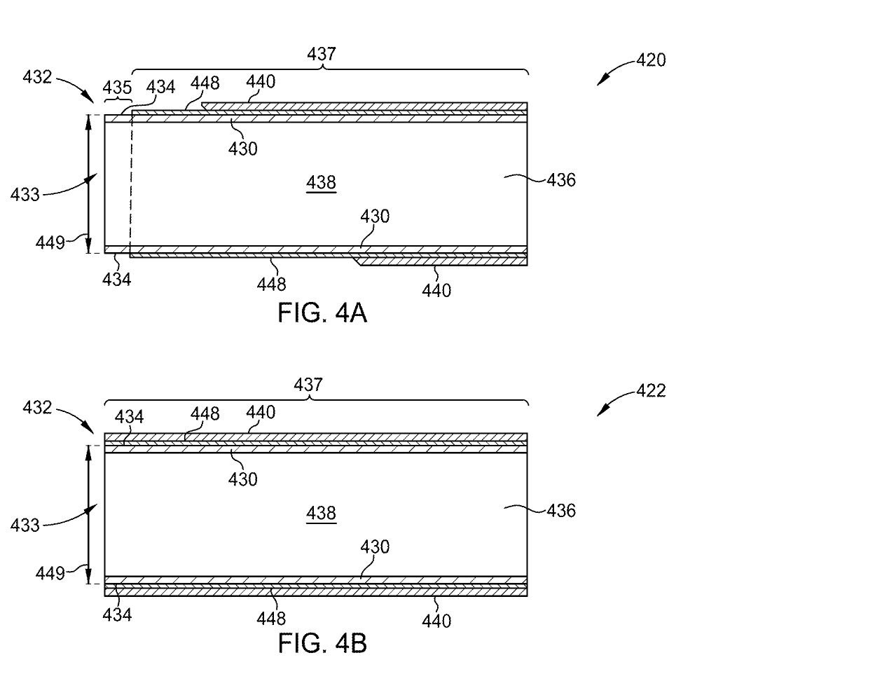

FIG. 4A depicts a left side cross-sectional view of the distal end of a tip 420 of a third example aspirating diathermy probe, in accordance with certain embodiments of the present disclosure.

Generally, the third example aspirating diathermy probe includes the same components as the aspirating diathermy probe 200, with the tip 220 replaced by the tip 420. Additionally, the tip 420 is the same as the tip 320 except for the angle of the distal end 432 of the aspiration tube 430.

In certain embodiments, the tip 420 includes, among other things, an aspiration tube 430, an outer tube 440, and an electrical insulating layer 448 disposed between the aspiration tube 430 and the outer tube 440. The distal end 432 of the aspiration tube 430 is parallel to the elevation axis 449and defines an opening 433.

The aspiration tube 430 is made from an electrically conductive material, and has an outer surface 434, and an inner surface 436 that defines an inner passageway 438. The aspiration tube 430 functions as one of the electrodes for bipolar diathermy.

The electrical insulating layer 448 covers most of the outer surface 434 (such as the insulated section 437) except for an exposed section 435 of the aspiration tube 430. Generally, the exposed section 435 of the outer surface 434, the edge of the aspiration tube 430, and a distal portion of the inner surface 436 provide one electrode surface for bipolar diathermy.

The outer tube 440 is made from an electrically conductive material, and functions as the other electrode for bipolar diathermy. The outer tube 440 does not extend to the end of the aspiration tube 430.

FIG. 4B depicts a left side cross-sectional view of the distal end of a tip 422 of a variation of the third example aspirating diathermy probe, in accordance with certain embodiments of the present disclosure.

Generally, the tip 422 is the same as the tip 322 except for the angle of the distal end 432 of the aspiration tube 430. The insulating layer 448 covers all of the outer surface 434, which extends the insulated section 437 to the end of the aspirating tube 430 and removes the exposed section 435. The edge of the aspiration tube 430 and a distal portion of the inner surface 436 provide one electrode surface for bipolar diathermy. The outer tube 440 also extends to the edge of the aspiration tube 430.

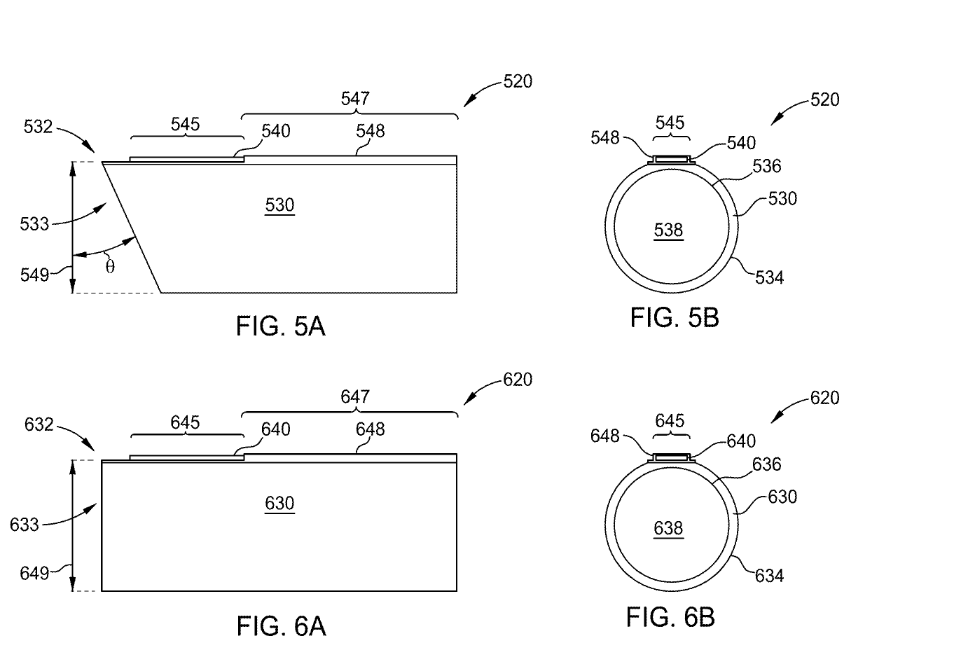

FIGS. 5A and 5B depict a left side view and a front view (respectively) of the distal end of a fourth example aspirating diathermy probe, in accordance with certain embodiments of the present disclosure. Generally, the fourth example aspirating diathermy probe includes the same components as the aspirating diathermy probe 200, with the tip 220 replaced by the tip 520.

In certain embodiments, the tip 520 includes, among other things, an aspiration tube 530, a conductor strip 540 disposed on the aspiration tube 530, and an electrical insulating layer 548 disposed between the aspiration tube 530 and the conductor strip 540. The distal end 532 of the aspiration tube 530 forms an angle θ with respect to the elevation axis 549 and defines an opening 533.

The aspiration tube 530 is made from electrically conductive material, and has an outer surface 534, and an inner surface 536 that defines an inner passageway 538. At least a portion of the inner surface 536 functions as one electrode for bipolar diathermy.

The electrical insulating layer 548 covers an upper portion of the outer surface 534. The electrical insulating layer 548 also covers most of the conductor strip 540 (such as the insulated section 547) except for an exposed section 545 of the conductor strip 540 which does not extend to the edge of the distal end 532.

The conductor strip 540 is made from an electrically conductive material and is electrically coupled to the other insulated electrical conductor of the diathermy electrical cable 260. The exposed section 545 of the conductor strip 540 forms the other electrode for bipolar diathermy.

The electrical insulating layer 548 and the conductor strip 540 may be applied to the aspiration tube 530 using various methods.

In certain embodiments, a flat surface or plane is machined along the axial length of the outer surface 534, and a first portion of the electrical insulating layer 548 is applied to the flat surface, such as by jet printing, etc. The conductor strip 540 is then applied to the electrical insulating layer 548, such as by jet printing, etc. A second portion of the electrical insulating layer 548 is then applied to the conductor strip 540, such as by jet printing, etc., leaving the exposed section 545 of the conductor strip 540 uninsulated. The exposed section 545 of the conductor strip 540 may form a rectangular shape (as depicted). Other shapes may also be used, such as triangular shapes, etc. Generally, the thickness of the first and second portions of the electrical insulating layer 548 and the conductor strip 540 may be about 4 to 6 microns, such as 5 microns. Other thicknesses may also be used, such as 6 to 10 microns, etc.

FIGS. 6A and 6B depict a left side view and a front view (respectively) of the distal end of a fifth example aspirating diathermy probe, in accordance with certain embodiments of the present disclosure. Generally, the fifth example aspirating diathermy probe includes the same components as the aspirating diathermy probe 200, with the tip 220 replaced by the tip 620. Additionally, the tip 620 is the same as the tip 520 except for the angle of the distal end 632 of the aspiration tube 630.

In certain embodiments, the tip 620 includes, among other things, an aspiration tube 630, a conductor strip 640 disposed on the aspiration tube 630, and an electrical insulating layer 648 disposed between the aspiration tube 630 and the conductor strip 640. The distal end 632 of the aspiration tube 630 is parallel to the elevation axis 649 and defines an opening 633.

The aspiration tube 630 is made from an electrically conductive material, and has an outer surface 634, and an inner surface 636 that defines an inner passageway 638. At least a portion of the inner surface 636 functions as one electrode for bipolar diathermy.

The electrical insulating layer 648 covers an upper portion of the outer surface 634. The electrical insulating layer 648 also covers most of the conductor strip 640 (such as the insulated section 647) except for an exposed section 645 of the conductor strip 640 which does not extend to the edge of the distal end 632.

The conductor strip 640 is made from an electrically conductive material, and the exposed section 645 of the conductor strip 640 forms the other electrode for bipolar diathermy.

The electrical insulating layer 648 and the conductor strip 640 may be applied to the aspiration tube 630 using various methods, as described above with respect to the electrical insulating layer 548 and the conductor strip 540.

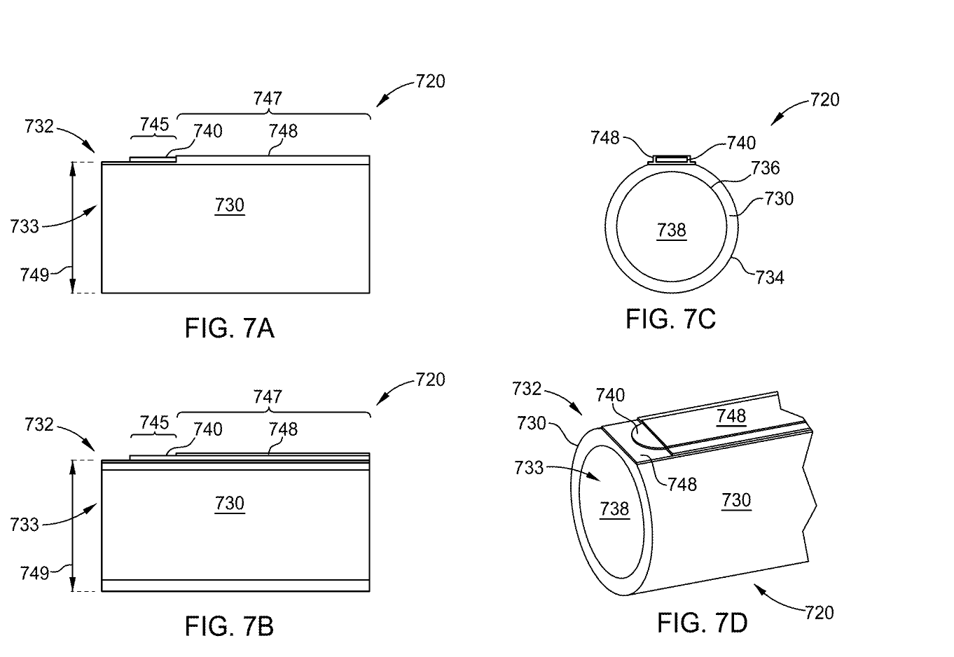

FIGS. 7A, 7B, 7C, and 7D depict views of the tip 720 of the sixth example aspirating diathermy probe, in accordance with certain embodiments of the present disclosure.

More particularly, FIG. 7A depicts a left side view of the distal end of the tip 720, FIG. 7B depicts a left side cross-sectional view of the distal end of the tip 720, FIG. 7C depicts a front view of the distal end of the tip 720, and FIG. 7D depicts a perspective view of the distal end of the tip 720. Generally, the sixth example aspirating diathermy probe includes the same components as the aspirating diathermy probe 200, with the tip 220 replaced by the tip 720. Additionally, the tip 720 is the same as the tip 620 except for the length and semicircular shape of the exposed section 745.

In certain embodiments, the tip 720 includes, among other things, an aspiration tube 730, a conductor strip 740 disposed on the aspiration tube 730, and an electrical insulating layer 748 disposed between the aspiration tube 730 and the conductor strip 740. The distal end 732 of the aspiration tube 730 is parallel to the elevation axis 749 and defines an opening 733.

The aspiration tube 730 is made from electrically conductive material, and has an outer surface 734, and an inner surface 736 that defines an inner passageway 738. At least a portion of the inner surface 736 functions as one electrode for bipolar diathermy.

The electrical insulating layer 748 covers an upper portion of the outer surface 734. The electrical insulating layer 748 also covers most of the conductor strip 740 (such as the insulated section 747) except for an exposed section 745 of the conductor strip 740 which does not extend to the edge of the distal end 732.

The conductor strip 740 is made from an electrically conductive material, and the exposed section 745 of the conductor strip 740 forms the other electrode for bipolar diathermy.

The electrical insulating layer 748 and the conductor strip 740 may be applied to the aspiration tube 730 using various methods, as described above with respect to the electrical insulating layer 548 and the conductor strip 540.

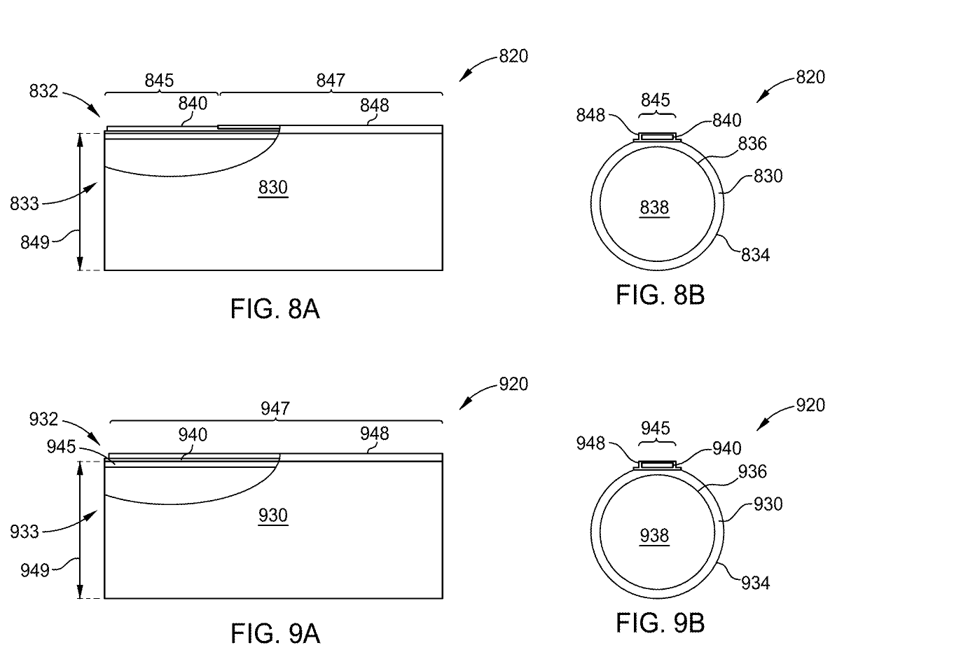

FIGS. 8A and 8B depict a left side view (partial cutaway) and a front view (respectively) of the distal end of a seventh example aspirating diathermy probe, in accordance with certain embodiments of the present disclosure. Generally, the seventh example aspirating diathermy probe includes the same components as the aspirating diathermy probe 200, with the tip 220 replaced by the tip 820. Additionally, the tip 820 is the same as the tip 620 except that the exposed section 845 extends to the edge of the distal end 832.

In certain embodiments, the tip 820 includes, among other things, an aspiration tube 830, a conductor strip 840 disposed on the aspiration tube 830, and an electrical insulating layer 848 disposed between the aspiration tube 830 and the conductor strip 840. The distal end 832 of the aspiration tube 830 is parallel to the elevation axis 849 and defines an opening 833.

The aspiration tube 830 is made from electrically conductive material, and has an outer surface 834, and an inner surface 836 that defines an inner passageway 838. At least a portion of the inner surface 836 functions as one electrode for bipolar diathermy.

The electrical insulating layer 848 covers an upper portion of the outer surface 834. The electrical insulating layer 848 also covers most of the conductor strip 840 (such as the insulated section 847) except for an exposed section 845 of the conductor strip 840 which extends to the edge of the distal end 832.

The conductor strip 840 is made from an electrically conductive material, and the exposed section 845 of the conductor strip 840 forms the other electrode for bipolar diathermy.

The electrical insulating layer 848 and the conductor strip 840 may be applied to the aspiration tube 830 using various methods, as described above with respect to the electrical insulating layer 548 and the conductor strip 540.

FIGS. 9A and 9B depict a left side view (partial cutaway) and a front view (respectively) of the distal end of an eighth example aspirating diathermy probe, in accordance with certain embodiments of the present disclosure. Generally, the eighth example aspirating diathermy probe includes the same components as the aspirating diathermy probe 200, with the tip 220 replaced by the tip 920. Additionally, the tip 920 is the same as the tip 820 except that the insulated section 947 extends to the edge of the distal end 932, and a small rectangular strip of the conductor strip 940 is uninsulated.

In certain embodiments, the tip 920 includes, among other things, an aspiration tube 930, a conductor strip 940 disposed on the aspiration tube 930, and an electrical insulating layer 948 disposed between the aspiration tube 930 and the conductor strip 940. The distal end 932 of the aspiration tube 930 is parallel to the elevation axis 949 and defines an opening 933.

The aspiration tube 930 is made from an electrically conductive material, and has an outer surface 934, and an inner surface 936 that defines an inner passageway 938. At least a portion of the inner surface 936 functions as one electrode for bipolar diathermy.

The electrical insulating layer 948 covers an upper portion of the outer surface 934. The electrical insulating layer 948 also covers the conductor strip 940 (such as the insulated section 947, which extends to the edge of the distal end 932). Only the end of the conductor strip 940 is exposed (such as the exposed section 945).

The conductor strip 940 is made from an electrically conductive material, and the exposed section 945 of the conductor strip 940 forms the other electrode for bipolar diathermy.

The electrical insulating layer 948 and the conductor strip 940 may be applied to the aspiration tube 930 using various methods, as described above with respect to the electrical insulating layer 548 and the conductor strip 540.

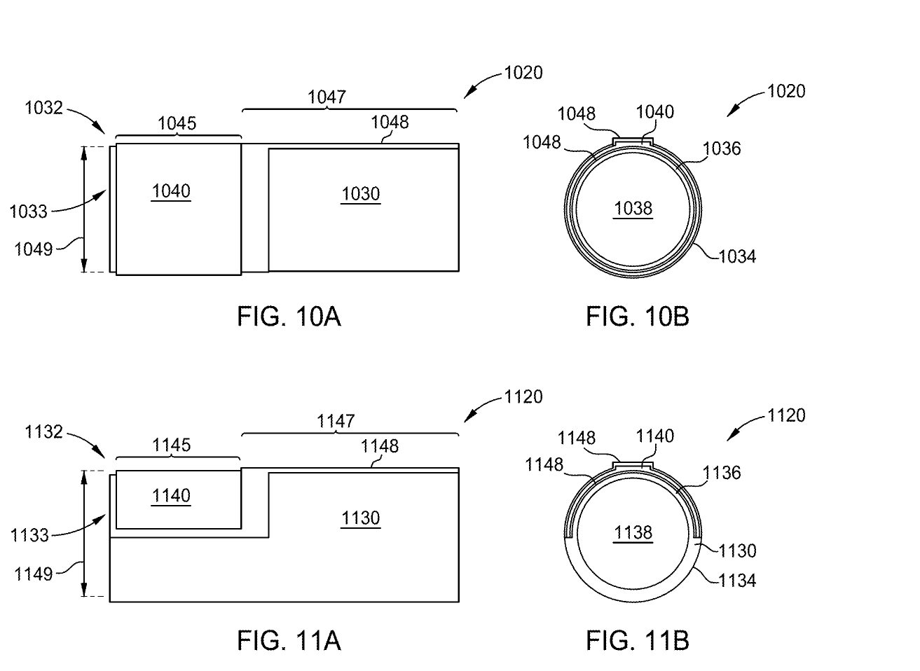

FIGS. 10A and 10B depict a left side view and a front view (respectively) of the distal end of a ninth example aspirating diathermy probe, in accordance with certain embodiments of the present disclosure. Generally, the ninth example aspirating diathermy probe includes the same components as the aspirating diathermy probe 200, with the tip 220 replaced by the tip 1020. Additionally, the tip 1020 is the same as the tip 620 with the addition of the ring-shaped surface of the conductor strip 1040 and the corresponding electrical insulating layer 1048 covering the distal end 1032 of the aspiration tube 1030.

In certain embodiments, the tip 1020 includes, among other things, an aspiration tube 1030, a conductor strip 1040 disposed on the aspiration tube 1030, and an electrical insulating layer 1048 disposed between the aspiration tube 1030 and the conductor strip 1040. The distal end 1032 of the aspiration tube 1030 is parallel to the elevation axis 1049 and defines an opening 1033.

The aspiration tube 1030 is made from electrically conductive material, and has an outer surface 1034, and an inner surface 1036 that defines an inner passageway 1038. At least a portion of the inner surface 1036 functions as one electrode for bipolar diathermy.

The electrical insulating layer 1048 covers an upper portion of the outer surface 1034 and the distal end 1032 of the aspiration tube 1030. The electrical insulating layer 1048 also covers the conductor strip 1040 (such as the insulated section 1047, which extends to the edge of the distal end 1032). A ring-shaped portion of the conductor strip 1040 is exposed (such as the exposed section 1045).

The conductor strip 1040 is made from an electrically conductive material, and the exposed section 1045 of the conductor strip 1040 forms the other electrode for bipolar diathermy.

The electrical insulating layer 1048 and the conductor strip 1040 may be applied to the aspiration tube 1030 using various methods, as described above with respect to the electrical insulating layer 548 and the conductor strip 540.

FIGS. 11A and 11B depict a left side view and a front view (respectively) of the distal end of a tenth example aspirating diathermy probe, in accordance with certain embodiments of the present disclosure. Generally, the tenth example aspirating diathermy probe includes the same components as the aspirating diathermy probe 200, with the tip 220 replaced by the tip 1120. Additionally, the tip 1120 is the same as the tip 1020 with a partial, ring-shaped portion of conductor strip 1140.

In certain embodiments, the tip 1120 includes, among other things, an aspiration tube 1130, a conductor strip 1140 disposed on the aspiration tube 1130, and an electrical insulating layer 1148 disposed between the aspiration tube 1130 and the conductor strip 1140. The distal end 1132 of the aspiration tube 1130 is parallel to the elevation axis 1149 and defines an opening 1133.

The aspiration tube 1130 is made from electrically conductive material, and has an outer surface 1134, and an inner surface 1136 that defines an inner passageway 1138. At least a portion of the inner surface 1136 functions as one electrode for bipolar diathermy.

The electrical insulating layer 1148 covers an upper portion of the outer surface 1134 and the distal end 1132 of the aspiration tube 1130. The electrical insulating layer 1148 also covers the conductor strip 1140 (such as the insulated section 1147). A partial ring-shaped portion of the conductor strip 1140 is exposed (such as the exposed section 1145).

The conductor strip 1140 is made from an electrically conductive material, and the exposed section 1145 of the conductor strip 1140 forms the other electrode for bipolar diathermy.

The electrical insulating layer 1148 and the conductor strip 1140 may be applied to the aspiration tube 1130 using various methods, as described above with respect to the electrical insulating layer 548 and the conductor strip 540.

FIG. 12A depicts the diathermy vitrectomy probe 1200, in accordance with certain embodiments of the present disclosure.

In certain embodiments, the diathermy vitrectomy probe 1200 includes, among other things, a handpiece 1210, a tip 1220, and a cable bundle 1250 that includes a diathermy electrical cable 1260, a flexible aspiration tube 1270, and one or more flexible pneumatic tubes 1280. The tip 1220 includes, among other things, an outer tube 1230 with a hollow bore or passageway and an outer port 1234, and a reciprocating inner tube 1290 (depicted in dotted line) with a hollow bore or passageway. The reciprocating inner tube 1290 is slidingly disposed within the passageway of the outer tube 1230 and is configured to cut and aspirate tissue in cooperation with the outer port 1234.

The handpiece 1210 has an outer gripping surface 1212 for the surgeon, and includes a passageway configured to support the distal portions of the diathermy electrical cable 1260, the flexible aspiration tube 1270, and the flexible pneumatic tubes 1280. The passageway extends from the proximal end of the handpiece 1210 to the tip 1220 attached to the distal end of the handpiece 1210. The handpiece 1210 also includes a pneumatic actuator coupled to the flexible pneumatic tubes 1280. In certain embodiments, the pneumatic actuator may be driven by separate air pressure sources provided by two flexible pneumatic tubes 1280. In other embodiments, the pneumatic actuator may be driven by a single air pressure source provided by one flexible pneumatic tube 1280.

The diathermy electrical cable 1260 is connected to an electrical connector that is coupled to the diathermy electrical connectors 122 (FIGS. 1A, 1C). The flexible aspiration tube 1270 is connected to a fluidic connector that is coupled to the aspiration port 132 (FIGS. 1A, 1C). The flexible pneumatic tubes 1280 are coupled to pneumatic connectors that are coupled to the vitrectomy probe pneumatic connectors 128 (FIGS. 1A, 1C). The diathermy electrical cable 1260 includes two insulated electrical conductors, such as insulated copper wires, insulated tungsten wires, etc. Each insulated electrical conductor is coupled to an electrical terminal in the electrical connector.

The footswitch 172 may be used to control the diathermy, cutting, and aspiration functions of the diathermy vitrectomy probe 1200.

In certain embodiments, the tip 1220 is removable and includes a mechanical connector to mechanically couple the reciprocating inner tube 1290 to the pneumatic actuator, a fluidic connector to couple the reciprocating inner tube 1290 to the flexible aspiration tube 1270, and electrical connectors to couple the outer tube 1230 and the conductor strip 1240 to the insulated electrical conductors of the diathermy electrical cable 1260.

In some embodiments, the cable bundle 1250 includes a flexible irrigating fluid tube, and the tip 1220 includes an irrigating fluid tube to deliver irrigating fluid to the eye 10. The flexible irrigating fluid tube is coupled to the irrigating fluid tube, and to a fluidic connector at the proximal end of the cable bundle 1250. The fluidic connector may be coupled to the irrigation port 134. Alternatively, the reciprocating inner tube 1290 may be coupled to a valve that selects between the flexible irrigating fluid tube and the flexible aspiration tube 270. The footswitch 172 may also be used to control the irrigation function of the aspirating diathermy probe 200.

FIGS. 12B and 12C depict a left side view and a front view (respectively) of the tip 1220 of the diathermy vitrectomy probe 1200, in accordance with certain embodiments of the present disclosure.

In certain embodiments, the tip 1220 includes the outer tube 1230, the reciprocating inner tube 1290, a conductor strip 1240 disposed on the outer tube 1230, and an electrical insulating layer 1248 disposed between the outer tube 1230 and the conductor strip 1240.

The outer tube 1230 has a distal portion 1232 with a closed end 1233 and the outer port 1234. The closed end 1233 forms an angle θ with respect to the elevation axis 1249. The outer tube 1230 is made from an electrically conductive material and is electrically coupled to one of the insulated conductors of the diathermy electrical cable 1260. The outer tube 1230 functions as one of the electrodes for bipolar diathermy.

The reciprocating inner tube 1290 has a hollow bore or passageway, and a distal portion that includes a cutting surface 1294 that defines an open end 1295. The reciprocating inner tube 1290 is mechanically coupled to the pneumatic actuator, and fluidically coupled to the flexible aspiration tube 1270. The pneumatic actuator causes the reciprocating inner tube 1290 to move back-and-forth within the passageway of the outer tube 1230, which causes the cutting surface 1294 to cut or slice tissue that protrudes into the outer port 1234. The tissue is aspirated into the open end 1295, through the passageway, to the distal end of the flexible aspiration tube 1270. In some embodiments, the distal portion of the reciprocating inner tube 1290 may include an additional cutting surface 1296 that defines an inner port 1297 through which tissue may be cut and aspirated.

The conductor strip 1240 is made from electrically conductive material, such as tungsten, copper, silver, stainless steel, etc. The conductor strip 1240 is electrically coupled to the other insulated electrical conductor of the diathermy electrical cable 260. The conductor strip 1240 functions as the other electrode for bipolar diathermy.

In certain embodiments, the electrical insulating layer 1248 partially covers the conductor strip 1240 to form an exposed section 1245 and insulated sections 1247. A first insulated section 1242 extends along a first side of the outer tube 1230, and a second insulated section 1244 extending along a second side of the outer tube 1230. The exposed section 1245 is located on the distal portion of the outer tube 1230, between the closed end 1233 and the outer port 1234. The electrical insulating layer 1248 is made from electrically insulating material.

More particularly, the distal portion 1232 of the outer tube 1230 (such as the closed end 1233 and the region proximate to the closed end 1233) forms a first diathermy electrode, and the exposed section 1245 of the conductor strip 1240 forms a second diathermy electrode.

The electrical insulating layer 1248 and the conductor strip 1240 may be applied to the outer tube 1230 using various methods, as described above with respect to the electrical insulating layer 548 and the conductor strip 540.

FIGS. 13A and 13B depict a left side view and a front view (respectively) of the tip 1320 of a second example diathermy vitrectomy probe, in accordance with certain embodiments of the present disclosure. Generally, the second example diathermy vitrectomy probe includes the same components as the diathermy vitrectomy probe 1200, with the tip 1220 replaced by the tip 1320. Additionally, the tip 1320 is the same as the tip 1220 with a semicircular conductor strip 1140 disposed on the closed end 1333 of the outer tube 1330.

In certain embodiments, the tip 1320 includes the outer tube 1330, the reciprocating inner tube 1390, a conductor strip 1340 disposed on the outer tube 1330, and an electrical insulating layer 1348 disposed between the outer tube 1330 and the conductor strip 1340.

The outer tube 1330 has a distal portion 1332 with a closed end 1333 and the outer port 1334. The closed end 1333 forms an angle θ with respect to the elevation axis 1349. The outer tube 1330 is made from an electrically conductive material, and functions as one of the electrodes for bipolar diathermy.

The reciprocating inner tube 1390 has a hollow bore or passageway, and a distal portion that includes a cutting surface 1394 that defines an open end 1395. In some embodiments, the distal portion of the reciprocating inner tube 1390 may include an additional cutting surface 1396 that defines an inner port 1397 through which tissue may be cut and aspirated.

The conductor strip 1340 is made from an electrically conductive material, and functions as the other electrode for bipolar diathermy.

In certain embodiments, the electrical insulating layer 1348 partially covers the conductor strip 1340 to form an exposed section 1345 and insulated sections 1347. A first insulated section 1342 extends along a bottom side of the outer tube 1330, and a second insulated section 1344 covers the closed end 1333 of the outer tube 1330. The exposed section 1345 is located on the closed end 1333 of the outer tube 1330.

More particularly, the distal portion 1332 of the outer tube 1330 (such as the region proximate to the closed end 1333) forms a first diathermy electrode, and the exposed section 1345 of the conductor strip 1340 forms a second diathermy electrode.

The electrical insulating layer 1348 and the conductor strip 1340 may be applied to the outer tube 1330 using various methods, as described above with respect to the electrical insulating layer 548 and the conductor strip 540.

In addition to the tips 1220, 1320, other patterns of conductor strips and electrical insulating layers may also be used. For example, variations of the embodiments depicted FIGS. 3A to 11B that accommodate the closed end 1233 and the outer port 1234 may be used.

FIG. 14A depicts the diathermy forceps 1400, in accordance with certain embodiments of the present disclosure.

In certain embodiments, the diathermy forceps 1400 includes, among other things, a pneumatic handpiece 1410, a tip 1420, and a cable 1450 that includes a diathermy electrical cable 1460 and a flexible pneumatic tube 1480. The tip 1420 includes, among other things, an extension tube 1430 and a forceps tool 1490.

The pneumatic handpiece 1410 has an outer gripping surface 1412 for the surgeon, and includes a passageway configured to support the distal portions of the diathermy electrical cable 1460 and the flexible pneumatic tube 1480. The passageway extends from the proximal end of the pneumatic handpiece 1410 to the tip 1420 attached to the distal end of the pneumatic handpiece 1410. The pneumatic handpiece 1410 also includes a pneumatic actuator that is coupled to the flexible pneumatic tube 1480 and the extension tube 1430. The pneumatic actuator is driven by an air pressure source provided by the flexible pneumatic tube 1480. The extension tube 1430 is configured to open and close the forceps tool 1490 when actuated by the pneumatic actuator.

The diathermy electrical cable 1460 is connected to an electrical connector that is coupled to the diathermy electrical connectors 122 (FIGS. 1A, 1C). The flexible pneumatic tube 1480 is coupled to a pneumatic connector that is coupled to the pneumatic connector 124 (FIGS. 1A, 1C). The diathermy electrical cable 1460 includes two insulated electrical conductors, such as insulated copper wires, insulated tungsten wires, etc. Each insulated electrical conductor is coupled to an electrical terminal in the electrical connector.

The footswitch 172 may be used to control the diathermy and grasping functions of the diathermy forceps 1400.

FIG. 14B depicts the diathermy forceps 1402, in accordance with certain embodiments of the present disclosure.

Generally, the diathermy forceps 1402 includes the same components as the diathermy forceps 1400, with the manual handpiece 1414 replacing the pneumatic handpiece 1410, and the pneumatic tube removed from the cable 1450. Rather than a pneumatic actuator, the manual handpiece 1414 includes a mechanical actuator that is coupled to the extension tube 1430. The mechanical actuator is activated by the surgeon, for example, by pressing and releasing the handpiece to grasp and release tissue in the forceps tool 1490.

FIG. 14C depicts a left side view of the tip 1420 of the diathermy forceps 1400, 1402, in accordance with certain embodiments of the present disclosure.

In certain embodiments, the tip 1420 includes the extension tube 1430 and the forceps tool 1490. The forceps tool 1490 includes a first jaw 1491 that has an arm 1492 and a grasping tip 1493, and a second jaw 1495 that has an arm 1496 and a grasping tip 1497 that opposes the grasping tip 1493. The arm 1492 and the arm 1496 are generally straight, while the grasping tip 1493 and the grasping tip 1497 have a curved shape, such as a 90° curve, etc. A conductor strip 1440 is disposed on the outer surface of the first jaw 1491, and an electrical insulating layer 1448 is disposed between the first jaw 1491 and the conductor strip 1440.

Generally, the forceps tool 1490 has an open position and a closed position. In the open position (depicted), the grasping tip 1493 and the grasping tip 1497 are displaced from one another to form a gap 1494 into which tissue may be maneuvered for grasping. In the closed position, the grasping tip 1493 and the grasping tip 1497 are in contact with, and apply pressure to, the tissue. In the absence of the tissue, the grasping tip 1493 and the grasping tip 1497 are in contact with one another.

In certain embodiments, the first jaw 1491 is made from an electrically conductive material, and functions as one of the electrodes for bipolar diathermy. In other embodiments, the second jaw 1495 is made from an electrically conductive material, and functions as one of the electrodes for bipolar diathermy.

The conductor strip 1440 is made from an electrically conductive material, and functions as the other electrode for bipolar diathermy.

In certain embodiments, the electrical insulating layer 1448 partially covers the first jaw 1491 to form an insulated section 1442 that is covered by the electrical insulating layer 1448 and an exposed section 1444 that is not covered by the electrical insulating layer 1448. Similarly, the electrical insulating layer 1448 partially covers the conductor strip 1440 to form an exposed section 1445 and insulated section 1447. The insulated section 1442 extends along the arm 1492 and encloses the grasping tip 1493. The exposed section 1445 is located on the outer surface of the grasping tip 1493.

More particularly, the exposed section 1444 of the arm 1492 forms the first diathermy electrode, and the exposed section 1445 of the conductor strip 1440 forms a second diathermy electrode.

The electrical insulating layer 1448 and the conductor strip 1440 may be applied to the extension tube 1430 using various methods, as described above with respect to the electrical insulating layer 548 and the conductor strip 540.

FIG. 15 depicts a left side view of the tip 1520 of the diathermy forceps 1400, 1402, in accordance with certain embodiments of the present disclosure.

Generally, the tip 1520 is the same as the tip 1420 with a straight grasping tip 1593 in place of the curved grasping tip 1493, and a straight grasping tip 1597 in place of the curved grasping tip 1497.

In certain embodiments, the tip 1520 includes the extension tube 1530 and the forceps tool 1590. The forceps tool 1590 includes a first jaw 1591 that has an arm 1592 and a grasping tip 1593, and a second jaw 1595 that has an arm 1596 and a grasping tip 1597 that opposes the grasping tip 1593. The arm 1592, the arm 1596, the grasping tip 1593, and the grasping tip 1597 are generally straight. A conductor strip 1540 is disposed on the outer surface of the first jaw 1591, and an electrical insulating layer 1548 is disposed between the first jaw 1591 and the conductor strip 1540.

Generally, the forceps tool 1590 has an open position and a closed position. In the open position (depicted), the grasping tip 1593 and the grasping tip 1597 are displaced from one another to form a gap 1594 into which tissue may be maneuvered for grasping. In the closed position, the grasping tip 1593 and the grasping tip 1597 are in contact with, and apply pressure to, the tissue. In the absence of the tissue, the grasping tip 1593 and the grasping tip 1597 are in contact with one another.

In certain embodiments, the first jaw 1591 is made from an electrically conductive material, and functions as one of the electrodes for bipolar diathermy. In other embodiments, the second jaw 1595 is made from an electrically conductive material, and functions as one of the electrodes for bipolar diathermy.

The conductor strip 1540 is made from an electrically conductive material, and functions as the other electrode for bipolar diathermy.

In certain embodiments, the electrical insulating layer 1548 partially covers the first jaw 1591 to form an insulated section 1542 that is covered by the electrical insulating layer 1548 and an exposed section 1544 that is not covered by the electrical insulating layer 1548. Similarly, the electrical insulating layer 1548 partially covers the conductor strip 1540 to form an exposed section 1545 and insulated section 1547. The insulated section 1542 extends along the arm 1592 to the grasping tip 1593. The exposed section 1545 is located on the outer surface of the grasping tip 1593.

More particularly, the exposed section 1544 of the arm 1592 forms the first diathermy electrode, and the exposed section 1545 of the conductor strip 1540 forms a second diathermy electrode.

The electrical insulating layer 1548 and the conductor strip 1540 may be applied to the extension tube 1530 using various methods, as described above with respect to the electrical insulating layer 548 and the conductor strip 540.

FIG. 16A depicts a diathermy scissors 1600, in accordance with certain embodiments of the present disclosure. Generally, the diathermy scissors 1600 includes the same components as the diathermy forceps 1400, with the tip 1420 replaced by the tip 1620.

In certain embodiments, the diathermy scissors 1600 includes, among other things, a handpiece 1610 with an outer gripping surface 1612 for the surgeon, a tip 1620, and a cable 1650 that includes a diathermy electrical cable 1660 and a flexible pneumatic tube 1680. The tip 1620 includes, among other things, an extension tube 1630 and a scissors tool 1690.

FIG. 16B depicts the diathermy scissors 1602, in accordance with certain embodiments of the present disclosure.

Generally, the diathermy scissors 1602 includes the same components as the diathermy scissors 1600, with the manual handpiece 1614 replacing the pneumatic handpiece 1610, and the pneumatic tube removed from the cable 1650. Rather than a pneumatic actuator, the manual handpiece 1614 includes a mechanical actuator that is coupled to the extension tube 1630. The mechanical actuator is activated by the surgeon, for example, by pressing and releasing the handpiece to cut tissue by the scissors tool 1690.

FIG. 16C depicts a left side view of the tip 1620 of the diathermy scissors 1600, 1602, in accordance with certain embodiments of the present disclosure.

In certain embodiments, the tip 1620 includes the extension tube 1630 and the scissors tool 1690. The scissors tool 1690 includes a first blade 1691, and a second blade 1695 that opposes the first blade 1691. The first blade 1691 and the second blade 1695 are generally curved. A conductor strip 1640 is disposed on the outer surface of the first blade 1691, and an electrical insulating layer 1648 is disposed between the first blade 1691 and the conductor strip 1640.

Generally, the scissors tool 1690 has an open position and a closed position. In the open position, the first blade 1691 and the second blade 1695 are displaced from one another to form a gap into which tissue may be maneuvered for cutting. In the closed position (depicted), the first blade 1691 and the second blade 1695 are in contact with one another. As the first blade 1691 and the second blade 1695 move from the open position to the closed position, the tissue present in the gap is progressively cut or sliced along the cutting surfaces of the first blade 1691 and the second blade 1695.

In certain embodiments, the first blade 1691 is made from electrically conductive material, and functions as one of the electrodes for bipolar diathermy. In other embodiments, the second blade 1695 is made from an electrically conductive material, and functions as one of the electrodes for bipolar diathermy.

The conductor strip 1640 is made from an electrically conductive material, and functions as the other electrode for bipolar diathermy.

In certain embodiments, the electrical insulating layer 1648 partially covers the first blade 1691 to form an insulated section 1642 that is covered by the electrical insulating layer 1648 and an exposed section 1644 that is not covered by the electrical insulating layer 1648. Similarly, the electrical insulating layer 1648 partially covers the conductor strip 1640 to form an exposed section 1645 and insulated section 1647. The insulated section 1642 extends along the first blade 1691 from the proximal end to the distal end. The exposed section 1645 is located on the outer surface of the distal end of the first blade 1691.

More particularly, the exposed section 1644 of the first blade 1691 forms the first diathermy electrode, and the exposed section 1645 of the conductor strip 1640 forms a second diathermy electrode.

The electrical insulating layer 1648 and the conductor strip 1640 may be applied to the first blade 1691 using various methods, as described above with respect to the electrical insulating layer 548 and the conductor strip 540.

FIG. 17 depicts a left side view of an alternate embodiment of the tip (tip 1720) of the diathermy scissors 1600, 1602, in accordance with certain embodiments of the present disclosure.

Generally, the tip 1720 is the same as the tip 1620 with a straight blade 1791 in place of the curved blade 1691, and a straight blade 1795 in place of the curved blade 1795.

In certain embodiments, the tip 1720 includes the extension tube 1730 and the scissors tool 1790. The scissors tool 1790 includes a first blade 1791, and a second blade 1795 that opposes the first blade 1791. The first blade 1791 and the second blade 1795 are generally straight. A conductor strip 1740 is disposed on the outer surface of the first blade 1791, and an electrical insulating layer 1748 is disposed between the first blade 1791 and the conductor strip 1740.

Generally, the scissors tool 1790 has an open position and a closed position. In the open position (depicted), the first blade 1791 and the second blade 1795 are displaced from one another to form a gap into which tissue may be maneuvered for cutting. In the closed position, the first blade 1791 and the second blade 1795 are in contact with one another. As the first blade 1791 and the second blade 1795 move from the open position to the closed position, the tissue present in the gap is progressively cut or sliced along the cutting surfaces of the first blade 1791 and the second blade 1795.

In certain embodiments, the first blade 1791 is made from an electrically conductive material, and functions as one of the electrodes for bipolar diathermy. In other embodiments, the second blade 1795 is made from an electrically conductive material, and functions as one of the electrodes for bipolar diathermy.

The conductor strip 1740 is made from an electrically conductive material, and functions as the other electrode for bipolar diathermy.

In certain embodiments, the electrical insulating layer 1748 partially covers the first blade 1791 to form an insulated section 1742 that is covered by the electrical insulating layer 1748 and an exposed section 1744 that is not covered by the electrical insulating layer 1748. Similarly, the electrical insulating layer 1748 partially covers the conductor strip 1740 to form an exposed section 1745 and insulated section 1747. The insulated section 1742 extends along the first blade 1791 from the proximal end to the distal end. The exposed section 1745 is located on the outer surface of the distal end of the first blade 1791.

More particularly, the exposed section 1744 of the first blade 1791 forms the first diathermy electrode, and the exposed section 1745 of the conductor strip 1740 forms a second diathermy electrode.

The electrical insulating layer 1748 and the conductor strip 1740 may be applied to the first blade 1791 using various methods, as described above with respect to the electrical insulating layer 548 and the conductor strip 540.

### Example Embodiments

In certain embodiments, a surgical device comprises a diathermy electrical cable, a flexible aspiration tube, at least one flexible pneumatic tube, a handpiece, and a tip attached to the handpiece. The handpiece comprises a pneumatic actuator coupled to a distal portion of the flexible pneumatic tube. The handpiece is configured to support a distal portion of the diathermy electrical cable, a distal portion of the flexible aspiration tube, and the distal portion of the flexible pneumatic tube. The tip comprises an outer tube, a reciprocating inner tube, a conductor strip disposed on the outer tube, and an electrical insulating layer disposed between the outer tube and the conductor strip. The outer tube comprises a passageway and a distal portion comprising a closed end and an outer port. The reciprocating inner tube comprises a passageway and a distal portion comprising a cutting surface defining an open end. The reciprocating inner tube is disposed within the passageway of the outer tube and is coupled to the pneumatic actuator and the distal portion of the flexible aspiration tube. The outer tube and the conductor strip are coupled to the distal portion of the diathermy electrical cable. The outer tube is a first diathermy electrode, and the conductor strip is a second diathermy electrode.

In certain embodiments, a surgical device comprises a diathermy electrical cable, a handpiece, and a tip attached to the handpiece. The handpiece comprises an actuator and is configured to support a distal portion of the diathermy electrical cable. The tip comprises a first jaw, a second jaw, an extension tube, a conductor strip, and an electrical insulating layer. The first jaw comprises a first grasping tip, and the second jaw comprises a second grasping tip. The extension tube is coupled to the actuator and is configured to open and close the first jaw and the second jaw. The conductor strip is disposed on the first jaw and is coupled to the distal portion of the diathermy electrical cable. The electrical insulating layer is disposed between the first jaw and the conductor strip. The first jaw is a first diathermy electrode, and the conductor strip is a second diathermy electrode.

In certain embodiments, a surgical device comprises a diathermy electrical cable, a handpiece, and a tip attached to the handpiece. The handpiece comprises an actuator and is configured to support a distal portion of the diathermy electrical cable. The tip comprises a first blade, a second blade, an extension tube, a conductor strip, and an electrical insulating layer. The first blade is coupled to the diathermy electrical cable. The extension tube is coupled to the actuator and is configured to open and close the first blade and the second blade. The conductor strip is disposed on the first blade. The electrical insulating layer is disposed between the first blade and the conductor strip. The first blade is a first diathermy electrode, and the conductor strip is a second diathermy electrode.

The many features and advantages of the disclosure are apparent from the detailed specification, and, thus, it is intended by the appended claims to cover all such features and advantages of the disclosure which fall within the scope of the disclosure. Further, since numerous modifications and variations will readily occur to those skilled in the art, it is not desired to limit the disclosure to the exact construction and operation illustrated and described, and, accordingly, all suitable modifications and equivalents may be resorted to that fall within the scope of the disclosure.

What is claimed is:

1. A surgical device, comprising:
a diathermy electrical cable;
a flexible aspiration tube;
at least one flexible pneumatic tube;
a handpiece comprising a pneumatic actuator coupled to a distal portion of the flexible pneumatic tube, the handpiece configured to support a distal portion of the diathermy electrical cable, a distal portion of the flexible aspiration tube, and the distal portion of the flexible pneumatic tube; and
a tip attached to the handpiece, the tip comprising:
an outer tube comprising a passageway and a distal portion comprising a closed end and an outer port, the outer tube coupled to the distal portion of the diathermy electrical cable,
a reciprocating inner tube comprising a passageway and a distal portion comprising a cutting surface defining an open end, the reciprocating inner tube disposed within the passageway of the outer tube, the reciprocating inner tube coupled to the pneumatic actuator and the distal portion of the flexible aspiration tube,
a conductor strip disposed on the outer tube, the conductor strip coupled to the distal portion of the diathermy electrical cable, and
an electrical insulating layer disposed between the outer tube and the conductor strip,
wherein:
the outer tube is a first diathermy electrode, and
the conductor strip is a second diathermy electrode.

2. The surgical device of claim 1, wherein:
the electrical insulating layer partially covers the conductor strip to form a first insulated section extending along a first side of the outer tube, a second insulated section extending along a second side of the outer tube, and an exposed section located on the distal portion of the outer tube;
the distal portion of the outer tube forms the first diathermy electrode; and
the exposed section of the conductor strip forms the second diathermy electrode.

3. The surgical device of claim 2, wherein the exposed section is located between the closed end and the outer port.

4. The surgical device of claim 3, wherein the exposed section of the conductor strip forms a partial ring around the distal portion of the outer tube.

5. The surgical device of claim 1, wherein:
the electrical insulating layer partially covers the conductor strip to form a first insulated section extending along a bottom of the outer tube, a second insulated section extending along the closed end of the outer tube, and an exposed section located on the closed end of the outer tube,
the distal portion of the outer tube forms the first diathermy electrode; and
the exposed section of the conductor strip forms the second diathermy electrode.

6. The surgical device of claim 5, wherein the exposed section of the conductor strip forms a semicircular region.

7. The surgical device of claim 2, wherein the exposed section is located at an end of the distal portion of the outer tube.

8. The surgical device of claim 1, wherein the distal portion of the reciprocating inner tube comprises an additional cutting surface defining an inner port.

9. A surgical device, comprising:
a diathermy electrical cable;
a handpiece comprising an actuator, the handpiece configured to support a distal portion of the diathermy electrical cable; and
a tip attached to the handpiece, the tip comprising:
a first jaw comprising a first grasping tip,
a second jaw comprising a second grasping tip,
an extension tube coupled to the actuator, the extension tube configured to open and close the first jaw and the second jaw,
a conductor strip disposed on the first jaw, the conductor strip coupled to the distal portion of the diathermy electrical cable, and
an electrical insulating layer disposed between the first jaw and the conductor strip,
wherein:
the first jaw is a first diathermy electrode, and
the conductor strip is a second diathermy electrode.

10. The surgical device of claim 9, further comprising:
a flexible pneumatic tube,
wherein the handpiece is further configured to support a distal portion of the flexible pneumatic tube, and
wherein the actuator is a pneumatic actuator coupled to the distal portion of the flexible pneumatic tube.

11. The surgical device of claim 9, wherein the actuator is a mechanical actuator.

12. The surgical device of claim 9, wherein:
the electrical insulating layer partially covers the first jaw to form an insulated section that is covered by the electrical insulating layer and an exposed section that is not covered by the electrical insulating layer;
the electrical insulating layer partially covers the conductor strip to form an insulated section that is covered by the electrical insulating layer and an exposed section that is not covered by the electrical insulating layer,
the exposed section of the first jaw forms the first diathermy electrode; and
the exposed section of the conductor strip forms the second diathermy electrode.

13. The surgical device of claim 12, wherein the exposed section of the conductor strip is located on the first grasping tip.

14. The surgical device of claim 13, wherein the electrical insulating layer has a thickness of about 4 to 6 microns, and the conductor strip has a thickness of about 4 to 6 microns.

15. A surgical device, comprising:
a diathermy electrical cable;
a handpiece comprising an actuator, the handpiece configured to support a distal portion of the diathermy electrical cable; and
a tip attached to the handpiece, the tip comprising:
a first blade coupled to the diathermy electrical cable,
a second blade,
an extension tube coupled to the actuator, the extension tube configured to open and close the first blade and the second blade,
a conductor strip disposed on the first blade, and
an electrical insulating layer disposed between the first blade and the conductor strip,
wherein:
the first blade is a first diathermy electrode, and
the conductor strip is a second diathermy electrode.

16. The surgical device of claim 15, further comprising:
a flexible pneumatic tube,
wherein the handpiece is further configured to support a distal portion of the flexible pneumatic tube, and
wherein the actuator is a pneumatic actuator coupled to the distal portion of the flexible pneumatic tube.

17. The surgical device of claim 15, wherein the actuator is a mechanical actuator.

18. The surgical device of claim 15, wherein:
the electrical insulating layer partially covers the first blade to form an insulated section that is covered by the electrical insulating layer and an exposed section that is not covered by the electrical insulating layer;
the electrical insulating layer partially covers the conductor strip to form an insulated section that is covered by the electrical insulating layer and an exposed section that is not covered by the electrical insulating layer,
the exposed section of the first blade forms the first diathermy electrode; and
the exposed section of the conductor strip forms the second diathermy electrode.

19. The surgical device of claim 18, wherein the exposed section of the conductor strip is located on a distal end of the first blade.

20. The surgical device of claim 19, wherein the electrical insulating layer has a thickness of about 4 to 6 microns, and the conductor strip has a thickness of about 4 to 6 microns.
